(19) United States
(12) Patent Application Publication
(10) Pub. No.: US 2022/0136053 A1
(43) Pub. Date: May 5, 2022

(54) METHODS AND COMPOSITIONS FOR DEVELOPING TARGET SPECIFIC EXOSOME AND GROWTH FACTOR PRODUCTS

(71) Applicant: DIRECT BIOLOGICS LLC, Austin, TX (US)

(72) Inventors: Kenneth Allen PETTINE, Fort Collins, CO (US); Timothy Alexander MOSELEY, Carlsbad, CA (US)

(21) Appl. No.: 17/427,192

(22) PCT Filed: Jan. 30, 2020
(86) PCT No.: PCT/US20/15982

§ 371 Date: Jul. 30, 2021

Related U.S. Application Data
(60) Provisional application No. 62/798,908, filed on Jan. 30, 2019.

(51) Int. Cl. C12Q 1/6883; C12N 5/0775; C12Q 1/6886; A61K 35/28

(52) U.S. Cl. CPC C12Q 1/6883; C12N 5/0666; C12Q 2600/124; A61K 35/28; C12Q 2600/156; C12Q 1/6886

(57) ABSTRACT

Disclosed are methods for making individualized mesenchymal stem cell growth factor and exosome compositions and methods of their use to treat skin disorders, hair loss, pain, rheumatoid arthritis, osteoporosis, autism, depression, biopolar disorder, anxiety, celiac disease, and cancer.

This application claims the benefit of US Provisional Application No. 62/798,908, filed on Jan. 30, 2019, which is incorporated herein by reference in its entirety.

### I. BACKGROUND

Humans share 99.9% of the exact same genetics, meaning that differences between humans is only contained in 0.1% of an individual's genetics. The small differences are contained in single nucleotide polymorphisms (SNPs). It is estimated that 6 to 20 of these SNPs are responsible for all the differences observed in race, ethnicity, skin color, skin type, eye color, and hair color, as well as, baldness. Recent studies have discovered that very specific SNP profiles can accurately predict who will develop some disease or conditions and who will not as well as who has said disease or condition. This is true in males and females.

Currently, there are few, if any, products that exist that are specific to a specific skin color or ethnicity as well as few, if any hair products that account for genetic factors responsible for greying hair or baldness. Similarly, there are no products that address genetic underpinnings associated with many diseases or conditions. Thus, conventional skin and hair products and treatments do not adequately address the biological differences found in a recipient subject.

Therefore, what is needed are new dermatological and cosmetic treatments, methods of making such treatments, that are specific to a person's skin color, hair color, baldness, as well as specific country or ethnicity, the treatment comprising targeted SNPs found in the DNA of an mesenchymal stem cell (MSC), keratinocyte, or melanocyte from a representative donor.

### II. SUMMARY

Disclosed are methods and compositions related to mesenchymal stem cell and exosome treatment compositions and methods of their use to treat baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer.

In one aspect, disclosed herein are methods for creating mesenchymal stem cell and exosome treatment composition for treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject, the method comprising a) identifying the hair, skin color, skin type, race, ethnicity of an end user; b) identifying a single nucleotide polymorphism (SNP) associated with the disease or disorder in the subject; c) obtaining mesenchymal stem cells (MSCs) from a targeted donor having the same hair type, color, and/or ethnicity as the subject but with a single nucleotide polymorphism (SNP) profile that indicates the donor will never experience disease or disorder; and d) preparing an MSC and exosome preparation from the obtained MSCs, wherein the MSC and exosome preparation is created by culturing MSCs in media comprising growth conditions sufficient to generate MSCs and exosomes that comprise and or secrete corrective SNPs and cytokines. In some aspect, the method further comprises identifying the type and amount of growth factors (proteomic analysis) in the MSC and exosome preparation (such as, for example characterizing the exosome RNA through methods such as sequencing).

Also disclosed herein are methods for creating mesenchymal stem cell and exosome treatment composition for treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject of any preceding aspect, wherein the SNP comprises one or more SNPs present in one or more genes encoding Interleukin 18 (IL-18)(such as, for example rs1946518 and/or rs187238), Androgen Receptor (AR)(such as, for example, Rs6152, Rs 2223841, and/or Rs2497938), Histone deacetylase (HDAC)-4 (HDAC4)(such as, for example, Rs9287638), HDAC9 (such as, for example, Rs2073963 and/or Rs2180439), Paired Box 1 (PAX1)(such as, for example, Rs1160312 and/or Rs6047844), Forkhead box A2 (FOXA2) (such as, for example, Rs1160312 and/or Rs6047844), TAR DNA Binding Protein (TARDBP)(such as, for example Rs12565727), Autism Susceptibility Gene 2 (AUTS2)(such as, for example Rs6945541), SET Binding Protein 1 (SETBP1)(such as, for example Rs10502861), 17q21.31 (such as, for example, Rs12373124), tumor necrosis factor receptor superfamily member 6B (TNFRSF6B) (such as, for example Rs6010620), Zinc finger CCCH-type with G patch domain-containing protein (ZGPAT) (such as, for example Rs6010620), P2X purinoceptor 7 (P2RX7)(such as, for example Rs17525809, Rs28360447, Rs7958311, Rs1718119, Rs2230912, Rs28360457, Rs2230911, and/or Rs1653624), Oxytocin Receptor (such as, for example Rs237887, Rs 2268491, Rs2254298, and/or Rs 7632287), non-coding RNA (ncRNA)(such as, for example Rs11669309, Rs2298075, and/or Rs10237038), long non-coding RNA (lncRNA)(such as, for example Rs8028149, Rs16868911, Rs11543230, Rs9309325, Rs12951337, Rs15428265, Rs12683158, and/or Rs6982502), human leukocyte antigen (HLA)(such as, for example, Rs2269706), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

In one aspect, disclosed herein are mesenchymal stem cell and exosome treatment compositions for use in treating a disease or disorder in an end user subject, the composition comprising: a) a composition base; and b) a mesenchymal stem cell and exosome preparation derived from a donor having the same hair type, hair color, skin type, skin color, race, and/or ethnicity as an end user but with a single nucleotide polymorphism (SNP) profile that indicates the donor will never disease or disorder suffered by the end user subject; wherein the MSC and exosome preparation comprises at least one member selected from the group consisting of cells or cell conditioned media cultured under normal hyperoxic culturing conditions and cells cultured under harsh wound healing conditions.

Also disclosed herein are mesenchymal stem cell and exosome treatment compositions of any preceding aspect, wherein the SNP comprises one or more SNPs present in one or more genes encoding Interleukin 18 (IL-18)(such as, for example rs1946518 and/or rs187238), Androgen Receptor (AR)(such as, for example, Rs6152, Rs 2223841, and/or Rs2497938), Histone deacetylase (HDAC)-4 (HDAC4)(such as, for example, Rs9287638), HDAC9 (such as, for example, Rs2073963 and/or Rs2180439), Paired Box 1 (PAX1)(such as, for example, Rs1160312 and/or Rs6047844), Forkhead box A2 (FOXA2) (such as, for example, Rs1160312 and/or Rs6047844), TAR DNA Binding Protein (TARDBP)(such as, for example Rs12565727), Autism Susceptibility Gene 2 (AUTS2)(such as, for example Rs6945541), SET Binding Protein 1 (SETBP1)(such as, for example Rs10502861), 17q21.31 (such as, for example, Rs12373124), tumor necrosis factor receptor superfamily member 6B (TNFRSF6B) (such as, for example Rs6010620), Zinc finger CCCH-type with G patch domain-containing protein (ZGPAT) (such as, for example Rs6010620), P2X purinoceptor 7 (P2RX7)(such as, for example Rs17525809, Rs28360447, Rs7958311, Rs1718119, Rs2230912, Rs28360457, Rs2230911, and/or Rs1653624), Oxytocin Receptor (such as, for example Rs237887, Rs 2268491, Rs2254298, and/or Rs 7632287), non-coding RNA (ncRNA)(such as, for example Rs11669309, Rs2298075, and/or Rs10237038), long non-coding RNA (lncRNA)(such as, for example Rs8028149, Rs16868911, Rs11543230, Rs9309325, Rs12951337, Rs15428265, Rs12683158, and/or Rs6982502), human leukocyte antigen (HLA)(such as, for example, Rs2269706), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

In one aspect, disclosed herein are methods of treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject comprising administering to a subject any of the mesenchymal stem cell and exosome treatment compositions of any preceding aspect.

For example, in one aspect disclosed herein are methods of treating, inhibiting, reducing, ameliorating, preventing, and/or reversing a disease or disorder of any preceding aspect, wherein the disease or disorder is baldness (such as, for example, male pattern baldness, androgenetic alopecia, alopecia areata, cicatricial alopecia, telogen effluvium, or female pattern baldness), and wherein single nucleotide polymorphism (SNP) associated with the baldness in the subject comprises one or more of the SNPs Rs6152, Rs 2223841, Rs2497938, Rs1160312, 6047844, Rs2180439, Rs2073963, Rs12565727, Rs9287638, Rs6945541, Rs12373124, Rs10502861, Rs187238, and/or Rs1946518.

Also disclosed herein are methods of treating, inhibiting, reducing, ameliorating, preventing, and/or reversing a disease or disorder of any preceding aspect, wherein the cancer comprises pancreatic cancer or breast cancer. In one aspect, disclosed herein are methods of treating, inhibiting, reducing, ameliorating, preventing, and/or reversing a disease or disorder of any preceding aspect, wherein the disease is breast cancer and the SNP associated with the breast cancer in the subject comprises the SNP Rs2298075. Also disclosed herein are methods of treating, inhibiting, reducing, ameliorating, preventing, and/or reversing a disease or disorder of any preceding aspect, wherein the disease is pancreatic cancer and the SNP associated with the pancreatic cancer in the subject comprises the SNP Rs10237038.

In one aspect, disclosed herein are methods of treating, inhibiting, reducing, ameliorating, preventing, and/or reversing a disease or disorder of any preceding aspect, wherein the disease or disorder comprises autism spectrum disorder and the SNP associated with the autism spectrum disorder comprise one or more of the SNPs Rs237887, Rs 2268491, Rs2254298, and/or Rs 7632287.

Also disclosed herein are methods of treating, inhibiting, reducing, ameliorating, preventing, and/or reversing a disease or disorder of any preceding aspect, wherein the disease or disorder comprises osteoporosis and the SNP associated with the osteoporosis comprises one or more of the SNPs Rs28360447, Rs28360457, Rs1718119, Rs2230911, and/or Rs1653624.

In one aspect, disclosed herein are methods of treating, inhibiting, reducing, ameliorating, preventing, and/or reversing a disease or disorder of any preceding aspect, wherein the disease or disorder comprises Rheumatoid Arthritis and the SNP associated with the Rheumatoid Arthritis comprises the SNP Rs2269706.

Also disclosed herein are methods of treating, inhibiting, reducing, ameliorating, preventing, and/or reversing a disease or disorder of any preceding aspect, wherein the disease or disorder comprises Multiple Sclerosis and the SNP associated with the Multiple Sclerosis comprises one or more of the SNPs Rs17525809 and/or Rs28360447.

In one aspect, disclosed herein are methods of treating, inhibiting, reducing, ameliorating, preventing, and/or reversing a disease or disorder of any preceding aspect, wherein the disease or disorder comprises chronic pain and the SNP associated with the chronic pain comprises one or more of the SNPs Rs28360447 and/or Rs7958311.

Also disclosed herein are methods of treating, inhibiting, reducing, ameliorating, preventing, and/or reversing a disease or disorder of any preceding aspect, wherein the disease or disorder comprises atopic dermatitis and the SNP associated with the atopic dermatitis comprises the SNP Rs6010620.

In one aspect, disclosed herein are methods of treating, inhibiting, reducing, ameliorating, preventing, and/or reversing a disease or disorder of any preceding aspect, wherein the disease or disorder comprises hypertension and the SNP associated with the hypertension comprises the SNP Rs11669309.

Also disclosed herein are methods of treating, inhibiting, reducing, ameliorating, preventing, and/or reversing a disease or disorder of any preceding aspect, wherein the disease or disorder comprises coronary heart disease and the SNP associated with the heart disease comprises the SNP Rs6982502.

In one aspect, disclosed herein are methods of treating, inhibiting, reducing, ameliorating, preventing, and/or reversing a disease or disorder of any preceding aspect, wherein the disease or disorder comprises type II diabetes and the SNP associated with the type II diabetes comprises the SNP Rs12683158.

Also disclosed herein are methods of treating, inhibiting, reducing, ameliorating, preventing, and/or reversing a disease or disorder of any preceding aspect, wherein the disease or disorder comprises celiac disease and the SNP associated with the celiac disease comprises the SNP Rs15428265.

In one aspect, disclosed herein are methods of treating, inhibiting, reducing, ameliorating, preventing, and/or reversing a disease or disorder of any preceding aspect, wherein the disease or disorder anxiety and the SNP associated with the anxiety comprises the SNP Rs1718119.

Also disclosed herein are methods of treating, inhibiting, reducing, ameliorating, preventing, and/or reversing a disease or disorder of any preceding aspect, wherein the disease or disorder comprises bipolar disorder and the SNP associated with the bipolar disorder comprises the SNP Rs2230912.

In one aspect, disclosed herein are methods of treating, inhibiting, reducing, ameliorating, preventing, and/or reversing a disease or disorder of any preceding aspect, wherein the disease or disorder comprises depression and the SNP associated with the depression comprises the SNP Rs8028149.

Also disclosed herein are methods of treating, inhibiting, reducing, ameliorating, preventing, and/or reversing a disease or disorder of any preceding aspect, wherein the disease or disorder comprises the one or more of the SNPs Rs16868911, Rs11543230, Rs9309325, and/or Rs12951337.

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “subject” is defined herein to include animals such as mammals, including, but not limited to, primates (e.g., humans), cows, horses, pigs, sheep, goats, dogs, cats, rabbits, rats, mice and the like. In some embodiments, the subject is a human.

“Administration” to a subject includes any route of introducing or delivering to a subject an agent. Administration can be carried out by any suitable route, including oral, topical, intravenous; subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrastemal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “decrease” can refer to any change that results in a smaller gene expression, protein production, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing,” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain (i.e., nociception) relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions; groups, etc. of these materials are disclosed that while specific reference of each various individual and collective combinations and permutation of these compounds may not be explicitly disclosed, each is specifically contemplated and described herein. For example, if a particular treatment composition (such as a composition comprising growth factors, exosomes, or proteins derives from MSC) is disclosed and discussed and a number of modifications that can be made to a number of molecules including the treatment composition (such as a composition comprising growth factors, exosomes, or proteins derives from MSC) are discussed, specifically contemplated is each and every combination and permutation of treatment composition (such as a composition comprising growth factors, exosomes, or proteins derives from MSC) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is now known that an individual's genotype has a role in the expression of and reaction to exosomes and growth factors. This recognition provides credence to the philosophy of personalized medicine utilizing responsive agents rather than a dose of recombinant proteins or autologous growth factors such as, platelet rich plasma (PRP). It is well known that laboratory growth conditions can greatly influence the quantity of produced growth factors and exosomes. Subjecting laboratory MSCs to stress conditions has been shown to dramatically increase the quantity of released growth factors and exosomes. studies evaluated the proteome of MSC growth factors and MSC derived exosomes from cells cultured under ideal growth conditions and under ischemic tissue simulated conditions to elucidate key angiogenic paracrine effectors present and potentially differentially expressed in these conditions. In total, 6,342 proteins were identified in MSC growth factors and 1,927 proteins in MSC derived exosomes from cells subjected to stressful growing conditions. There was a substantial increase in the amount and number of different types of growth factors and exosomes produced by the cells grown under stress conditions versus those grown under ideal conditions.

Multilayered analyses identified several growth factors and exosomes that stimulate angiogenesis that were markedly increased in expression in MSCs exposed to simulated stress ischemic conditions; these growth factor proteins include platelet derived growth factor, epidermal growth factor, fibroblast growth factor, and most notably nuclear factor-kappaB (NFkB) signaling pathway proteins. Collectively, the results of the previous studies' proteomic analysis show that MSC derived growth factors and exosomes contain a robust profile of angiogenic paracrine effectors, which have potential for the treatment of numerous diseases.

In one aspect, disclosed herein are treatment compositions specific to a subject's body characteristics (such as, for example, skin color, skin type, ethnicity, race, hair type, and/or hair color) for treating, inhibiting, reducing, preventing and/or reversing baldness, hair greying, erectile dysfunction, and/or skin disorders. As disclosed herein, the treatment composition can comprise concentrated growth factors, exosomes, extracellular proteins, proteoglycans, cytokines, chemokines, proteins, and peptides derived from MSCs or similar fibroblast-like cells, keratinocytes or melanocytes, wherein the cells may be obtained from bone marrow, adipose (fat) stromal vascular fraction (SVF), bone, or other tissue sources before or after cell expansion. In some aspect, the treatment composition can further comprise stem cell factors (SCF).

Exosomes are small (30-200 nm) membrane-bound vesicles that are released into the extracellular milieu. Exosomes contain growth factors, signaling lipids, and various types of RNA inducing messenger RNA (mRNA) and microRNA (miRNA). Their RNA contents mediate many, if not most, of the effects on the cells with which the exosomes communicate. The RNA is placed into an exosome along with numerous peptide growth factors and signaling lipids by the Golgi bodies within the donor MSC. The exact type and amount of growth factor proteins, signaling lipids, and RNA placed into an exosome are dependent on the surrounding microenvironment and signals that are exposed to the MSC.

Accordingly, disclosed herein are methods for creating mesenchymal stem cell and exosome treatment composition for treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject, the method comprising a) identifying the hair, skin color, skin type, race, ethnicity of an end user; b) identifying a single nucleotide polymorphism (SNP) associated with the disease or disorder in the subject; c) obtaining mesenchymal stem cells (MSCs) from a targeted donor having the same hair type, color, and/or ethnicity as the subject but with a single nucleotide polymorphism (SNP) profile that indicates the donor will never experience disease or disorder; and d) preparing an MSC and exosome preparation from the obtained MSCs, wherein the MSC and exosome preparation is created by culturing MSCs in media comprising growth conditions sufficient to generate MSCs and exosomes that comprise and or secrete corrective SNPs and cytokines.

In some aspect, the method further comprises identifying the type and amount of growth factors in the MSC and exosome preparation. Analysis of the growth factors and the exosome preparation can involve any means known in the art for analyzing proteins and nucleic acids. It is understood that growth factors are primarily protein and peptides. Thus, the growth factors can be analyzed using any means of proteomic analysis known in the art, including but not limited to mass spectrometry, protein array, protein chips, enzyme-linked immunosorbent assay (ELISA), flow cytometry, enzyme-linked immunospot (ELISpot), SDS-PAGE, Mass spectrometric immunoassay (MSIA), electrospray ionization, matrix-assisted laser desorption/ionization (MALDI), western blot, and/or chromatorgraphy. As noted above in addition to proteomic cargo exosomes carry RNA which has a significant effect on target cells. Proteomic cargo of exosomes can be detected using the same techniques used to detect growth factors. Nucleic acid contained in exosomes can be characterized by any technique useful in the detection and identification of nucleic acid, including but not limited to sequencing, microarray, PCR, and northern blot.

Some embodiments of the present disclosure include a method for creating mesenchymal stem cell and exosome treatment composition for a specific pathology, unique complexion challenges, race, or ethnicity. The method may include identifying a single nucleotide polymorphism (SNP) of a potential donor to match that of an end user; obtaining mesenchymal skin cells (MSCs) from the donor having the same or a similar SNP profile as the end user; and preparing an MSC growth factor and exosome preparation from the obtained MSCs. The MSC preparation may be created by altering the growth conditions to create a specific product to match the specific pathology of the recipient by thorough characterization and proteomic analysis of the growth factors and molecular characterization of the exosome RNA present in the growth media to maximize the treatment efficacy by matching the product to the exact pathology identified and needing to be treated.

As noted above, it is understood and herein contemplated that the disclosed methods depend on the detection and use of SNPs associated with a disease state and the use of a donor with similar race, hair, eye, skin, and/or ethnic characteristics as the recipient but differing at the SNP for disclosed methods of making creating mesenchymal stem cell and exosome treatment composition. It is understood and herein contemplated that the SNP used will depend on the disease or disorder targeted for treatment as well as the end user subject being treated (said subject having to differ at the SNP from the donor). Accordingly, in one aspect herein are methods for creating mesenchymal stem cell and exosome treatment composition for treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject, wherein the disease or disorder is baldness (such as, for example, male pattern baldness, androgenetic alopecia, alopecia areata, cicatricial alopecia, telogen effluvium, or female pattern baldness), and wherein single nucleotide polymorphism (SNP) associated with the baldness in the subject comprises one or more of the SNPs Rs6152, Rs 2223841, Rs2497938, Rs1160312, 6047844, Rs2180439, Rs2073963, Rs12565727, Rs9287638, Rs6945541, Rs12373124, Rs10502861, Rs187238, and/or Rs1946518. Similarly, in one aspect, disclosed herein are methods for creating mesenchymal stem cell and exosome treatment composition for treating, inhibiting, reducing, ameliorating, preventing, and/or reversing autism spectrum disorder wherein the SNP associated with the autism spectrum disorder comprise one or more of the SNPs Rs237887, Rs 2268491, Rs2254298, and/or Rs 7632287. Also disclosed herein are methods for creating mesenchymal stem cell and exosome treatment composition for treating, inhibiting, reducing, ameliorating, preventing, and/or reversing osteoporosis wherein the SNP associated with the osteoporosis comprises one or more of the SNPs Rs28360447, Rs28360457, Rs1718119, Rs2230911, and/or Rs1653624. In one aspect, disclosed herein are methods for creating mesenchymal stem cell and exosome treatment composition for treating, inhibiting, reducing, ameliorating, preventing, and/or reversing Rheumatoid Arthritis wherein the SNP associated with the Rheumatoid Arthritis comprises the SNP Rs2269706. Also disclosed herein are methods for creating mesenchymal stem cell and exosome treatment composition for treating, inhibiting, reducing, ameliorating, preventing, and/or reversing Multiple Sclerosis wherein the SNP associated with the Multiple Sclerosis comprises one or more of the SNPs Rs17525809 and/or Rs28360447. In one aspect, disclosed herein are methods for creating mesenchymal stem cell and exosome treatment composition for treating, inhibiting, reducing, ameliorating, preventing, and/or reversing chronic pain wherein the SNP associated with the chronic pain comprises one or more of the SNPs Rs28360447 and/or Rs7958311. Also disclosed herein are methods for creating mesenchymal stem cell and exosome treatment composition for treating, inhibiting, reducing, ameliorating, preventing, and/or reversing atopic dermatitis comprises the SNP Rs6010620. In one aspect, disclosed herein are methods for creating mesenchymal stem cell and exosome treatment composition for treating, inhibiting, reducing, ameliorating, preventing, and/or reversing hypertension wherein the SNP associated with the hypertension comprises the SNP Rs11669309. Also disclosed herein are methods for creating mesenchymal stem cell and exosome treatment composition for treating, inhibiting, reducing, ameliorating, preventing, and/or reversing coronary heart disease wherein the SNP associated with the heart disease comprises the SNP Rs6982502. In one aspect, disclosed herein are methods for creating mesenchymal stem cell and exosome treatment composition for treating, inhibiting, reducing, ameliorating, preventing, and/or reversing type II diabetes and the SNP associated with type II diabetes comprises Rs12683158. Also disclosed herein are methods for creating mesenchymal stem cell and exosome treatment composition for treating, inhibiting, reducing, ameliorating, preventing, and/or reversing celiac disease wherein the SNP associated with the celiac disease comprises the SNP Rs15428265. In one aspect, disclosed herein are methods for creating mesenchymal stem cell and exosome treatment composition for treating, inhibiting, reducing, ameliorating, preventing, and/or reversing anxiety wherein the SNP associated with the anxiety comprises the SNP Rs1718119. Also disclosed herein are methods for creating mesenchymal stem cell and exosome treatment composition for treating, inhibiting, reducing, ameliorating, preventing, and/or reversing bipolar disorder wherein the SNP associated with the bipolar disorder comprises the SNP Rs2230912. In one aspect, disclosed herein are methods for creating mesenchymal stem cell and exosome treatment composition for treating, inhibiting, reducing, ameliorating, preventing, and/or reversing depression wherein the SNP associated with the depression comprises the SNP Rs8028149. Also disclosed herein are methods for creating mesenchymal stem cell and exosome treatment composition for treating, inhibiting, reducing, ameliorating, preventing, and/or reversing disease or disorder wherein the disease or disorder comprises the one or more of the SNPs Rs16868911, Rs11543230, Rs9309325, and/or Rs12951337.

In one aspect, it is understood and herein contemplated that the disclosed methods for creating mesenchymal stem cell and exosome treatment compositions can be used to generated MSC and exosome compositions for the treatment of treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Thus, in one aspect, disclosed herein are methods creating mesenchymal stem cell and exosome treatment composition for treating, inhibiting, reducing, ameliorating, preventing, and/or reversing a cancer, wherein the cancer comprises pancreatic cancer or breast cancer. In one aspect, disclosed herein are methods of creating mesenchymal stem cell and exosome treatment composition for treating, inhibiting, reducing, ameliorating, preventing, and/or reversing breast cancer wherein the SNP associated with the breast cancer in the subject comprises the SNP Rs2298075. Also disclosed herein are methods creating mesenchymal stem cell and exosome treatment composition for treating, inhibiting, reducing, ameliorating, preventing, and/or reversing pancreatic cancer wherein the SNP associated with the pancreatic cancer in the subject comprises the SNP Rs10237038.

It is understood and herein contemplated that the disclosed SNPs are associated with point mutations in the nucleic acid of particular genes. Thus, in one aspect, disclosed herein are methods for creating mesenchymal stem cell and exosome treatment composition for treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject, wherein the SNP comprises one or more SNPs present in one or more genes encoding Interleukin 18 (IL-18)(such as, for example rs1946518 and/or r5187238), Androgen Receptor (AR)(such as, for example, Rs6152, Rs 2223841, and/or Rs2497938), Histone deacetylase (HDAC)-4 (HDAC4)(such as, for example, Rs9287638), HDAC9 (such as, for example, Rs2073963 and/or Rs2180439), Paired Box 1 (PAX1)(such as, for example, Rs1160312 and/or Rs6047844), Forkhead box A2 (FOXA2) (such as, for example, Rs1160312 and/or Rs6047844), TAR DNA Binding Protein (TARDBP)(such as, for example Rs12565727), Autism Susceptibility Gene 2 (AUTS2)(such as, for example Rs6945541), SET Binding Protein 1 (SETBP1)(such as, for example Rs10502861), 17q21.31 (such as, for example, Rs12373124), tumor necrosis factor receptor superfamily member 6B (TNFRSF6B) (such as, for example Rs6010620), Zinc finger CCCH-type with G patch domain-containing protein (ZGPAT) (such as, for example Rs6010620), P2X purinoceptor 7 (P2RX7)(such as, for example Rs17525809, Rs28360447, Rs7958311, Rs1718119, Rs2230912, Rs28360457, Rs2230911, and/or Rs1653624), Oxytocin Receptor (such as, for example Rs237887, Rs 2268491, Rs2254298, and/or Rs 7632287), non-coding RNA (ncRNA)(such as, for example Rs11669309, Rs2298075, and/or Rs10237038), long non-coding RNA (lncRNA)(such as, for example Rs8028149, Rs16868911, Rs11543230, Rs9309325, Rs12951337, Rs15428265, Rs12683158, and/or Rs6982502), human leukocyte antigen (HLA)(such as, for example, Rs2269706), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

It is understood and herein contemplated that the disclosed methods result in mesenchymal stem cell (MSC) and exosome treatment compositions for use in treating a disease or disorder in a subject. Thus, in one aspect, disclosed herein are mesenchymal stem cell and exosome treatment compositions for use in treating a disease or disorder in an end user subject, the composition comprising: a) a composition base; and b) a mesenchymal stem cell and exosome preparation derived from a donor having the same hair type, hair color, hair type, skin type, skin color, race, and/or ethnicity as an end user but with a single nucleotide polymorphism (SNP) profile that indicates the donor will never disease or disorder suffered by the end user subject; wherein the MSC and exosome preparation comprises at least one member selected from the group consisting of cells or cell conditioned media cultured under normal hyperoxic culturing conditions and cells cultured under harsh wound healing conditions.

As noted above, the SNPs that distinguish the donor and the recipient can come from SNPs associated with any number of genes associated with a disease or disorder state. Thus, in one aspect, disclosed herein are mesenchymal stem cell and exosome treatment compositions, wherein the SNP comprises one or more SNPs present in one or more genes encoding Interleukin 18 (IL-18)(such as, for example rs1946518 and/or rs187238), Androgen Receptor (AR)(such as, for example, Rs6152, Rs 2223841, and/or Rs2497938), Histone deacetylase (HDAC)-4 (HDAC4)(such as, for example, Rs9287638), HDAC9 (such as, for example, Rs2073963 and/or Rs2180439), Paired Box 1 (PAX1)(such as, for example, Rs1160312 and/or Rs6047844), Forkhead box A2 (FOXA2) (such as, for example, Rs1160312 and/or Rs6047844), TAR DNA Binding Protein (TARDBP)(such as, for example Rs12565727), Autism Susceptibility Gene 2 (AUTS2)(such as, for example Rs6945541), SET Binding Protein 1 (SETBP1)(such as, for example Rs10502861), 17q21.31 (such as, for example, Rs12373124), tumor necrosis factor receptor superfamily member 6B (TNFRSF6B) (such as, for example Rs6010620), Zinc finger CCCH-type with G patch domain-containing protein (ZGPAT) (such as, for example Rs6010620), P2X purinoceptor 7 (P2RX7)(such as, for example Rs17525809, Rs28360447, Rs7958311, Rs1718119, Rs2230912, Rs28360457, Rs2230911, and/or Rs1653624), Oxytocin Receptor (such as, for example Rs237887, Rs 2268491, Rs2254298, and/or Rs 7632287), non-coding RNA (ncRNA)(such as, for example Rs11669309, Rs2298075, and/or Rs10237038), long non-coding RNA (lncRNA)(such as, for example Rs8028149, Rs16868911, Rs11543230, Rs9309325, Rs12951337, Rs15428265, Rs12683158, and/or Rs6982502), human leukocyte antigen (HLA)(such as, for example, Rs2269706), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

In embodiments, the treatment composition may comprise a base, and an MSC, keratinocyte, and/or melanocyte growth factor powder preparation, wherein the MSC preparation (MSC/K/M/Prep) may include at least one member selected from the group consisting of cells or cell conditioned media cultured under normal hyperoxic culturing conditions and cells cultured under harsh wound healing conditions. Hyperoxic culturing conditions may be defined as about 21%, wherein about 21% may be 21%±5%, oxygen with serum supplements and oxygen, while wound healing conditions may be defined as about 1 to about 5% oxygen in the presence of inflammatory cytokines, angiogenic factors, and/or reduced glucose.

The MSC/KM/Prep may comprise either conditioned media or lysate from cell culture expanded MSCs, keratinocytes, or melanocytes. In some embodiments, the composition may further comprise from about 0.01 to about 10 wt. % of a cell-free medium conditioned by growth of MSC/K/M/PREP or lineage cells, wherein the cells are cultured under normal hyperoxic culturing conditions or under wound healing conditions.

The MSC/KM/PREP conditioned media, lysates, and derived products or combinations thereof, optionally with other active ingredients, may be dissolved, mixed, or suspended in a mixture of emulsifying lanolin alcohols, waxes, and oils or a mixture of petrolatum or mineral oil, a quaternary ammonium compound, a fatty alcohol, and a fatty ester emollient, or lotions that are substantially similar in composition.

The base of the composition of the present disclosure may be any suitable or desired base, such as a lotion, a cream, a pigment, a serum, an oil, a gel, a hydrogel, a powder, a foundation, a facial mask, a lip care product, a hair car product, a hair car product, a skin cleanser, an exfoliant, an ointment, injectable, or the like. Alternatively, the base may comprise a material suitable for injection directly into or application directly onto a subject or any tissue, organ, or system of said subject.

In embodiments, the base may comprise a lotion comprising a mixture of emulsifying lanolin alcohols, waxes, and oils or a mixture of petrolatum or mineral oil, a quaternary ammonium compound, a fatty alcohol, and a fatty ester emollient. Alternatively, the base may comprise a cream comprising a mixture of emulsifying lanolin alcohols, water, petrolatum, glycerin, isostearyl palmitate, butylene glycol, glyceryl stearate, or a mixture thereof.

In some embodiments, the cosmetic base may be a carrier that may contain, for example, about 1 to about 20 wt. % of a humectant, about 0.1 to about 10 wt. % of a thickener and water. Alternatively, the carrier may comprise about 70 to about 99 wt. % of a surfactant, and about 0 to about 20 wt. % of a fat. The carrier may alternatively comprise about 80 to 99.9% of a thickener; about 5 to about 15% of a surfactant, about 2 to about 15% of a humectant, about 0 to about 80% of an oil, very small (<2%) amounts of preservative, coloring agent and/or perfume, and water if desired.

In embodiments, the composition may further comprise a penetration enhancer to improve epidermal penetration of the bioactive substance. Suitable penetration enhancers may include dimethyl sulfoxide (DMSO), DMSO-like compounds, ethanolic compounds, pyroglutamic acid esters, and the like. The composition may also include a sunscreen, anti-acne agents, anticellulite agents, and other additional components.

The composition may be filter-sterilized or concentrated. Moreover, the composition may be free from non-human animal products or may be derived from animal sources.

While the composition is described above as including MSCs, the use of other fibroblast-like cells is envisioned. The product may contain keratinocytes or melanocytes. The MSCs may be derived from multiple sources such as bone marrow stroma, adipose, blood, dermis, periosteum, bone, and other tissues. In embodiments, the MSCs may be derived from the patient to which the composition will be applied (autologous) or derived from another individual (allogeneic). The MSCs/K/M/PREP may be culture expanded to collect the conditioned media or to increase the quantity of cells for the lysate or used freshly prior to incorporation into the composition of the present disclosure.

Producing the treatment composition of the present disclosure may include first identifying the target consumer's race, ethnicity, skin color, skin type, eye color, and hair color, as well as, baldness characteristics, sourcing MSC's and exosomes from an individual with similar race, ethnicity, skin color, skin type, eye color, and hair color, as well as, baldness characteristics, and using the sourced MSC's to create a topical and/or injectable treatment to be applied to the subject.

As such, the final treatment composition may be customized for the end user based on hair characteristics. To use the hair and scalp treatment composition of the present disclosure, a user may simply apply the composition topically to the scalp. Alternatively, the composition may be injected directly into the dermis in areas affected by miniaturization. Administration can be as little as 0.1 mL to as much as 100 mL as appropriate for the given indication. For example, the administered dose can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 mL in total single administration or aliquoted over multiple injections. For example, a single 1 mL volume can be dispersed in 0.1 mL injections. When multiple injections are given, the volume of each injection can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, 10 mL. Injections can be made as a single site of injection or over 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900, or 1000 separate injections. Where multiple injections are used in an effected area, the distance between injection can be 0.1, 0.2, 0.25, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 6, 7, 8, 9, or 10 cm apart. While desired, it is understood and herein contemplated that a single treatment administration may not be sufficient to achieve the desired therapeutic results. Accordingly, administration can occur at a single time or 2, 3, 4, 5, 6, 7, 8, 910, 11, 12, 13, 14, 15, 16, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700, 800, 900, or 1000 separate times for the life of the host or duration of the treatment. When multiple administrations are made, administration can occur one time every 6, 12, 18, 24, 36, 48, 60, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 42, 49, 56, 58, 59, 60, 61, 62 days, 9, 10, 11, 12, 13, 14, 15, 16 weeks, 5, 6, 7, 8, 9, 10, 11, 12, 18, 24 months, 3, 4, 5, 6, 7, 8, 9, or 10 years.

It is understood and herein contemplated that the concentrated growth factors, exosomes, extracellular proteins, proteoglycans, cytokines, chemokines, proteins, and peptides derived from MSCs or similar fibroblast-like cells, keratinocytes or melanocytes used in the disclosed treatment compositions can be diluted to reach an administered dose. Diluents can be any suitable substance including but not limited to saline or any pharmaceutical based carrier or excipient disclosed herein. The dilution of the growth factors, exosomes, extracellular proteins, proteoglycans, cytokines, chemokines, proteins, and peptides derived from MSCs or similar: 1fibroblast-like cells, keratinocytes or melanocytes can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50 or 1:100.

As stated repeatedly throughout, the disclosure, the disclosed methods of generating a MSC and exosome treatment composition do, in fact, generate a MSC and exosome treatment composition that can be used in treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject. Thus, in one aspect, disclosed herein are methods of treating, inhibiting, reducing, ameliorating, preventing, and/or reversing a disease or disorder (such as, for example baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer) in a subject comprising administering to a subject any of the mesenchymal stem cell and exosome treatment compositions disclosed herein.

The utility of exosomes as a carrier system to deliver genetic correction has widespread applicability. Essentially any genetic disease resultant from minor sequence differences, for instance, single nucleotide polymorphisms (SNPs), could receive molecular modification proteins or nucleic acids via exosomic integration into target cells. These molecules may be introduced into exosomes from genetically modified cell lines or alternatively may be sourced from cells of individual donors who carry the non-disease associated genetic sequences.

The following examples of candidate SNPs associated with different disease states are provided as proof of concept for the described invention. The invention may be used to transport virtually any SNP corrective sequence and the scope of the invention's utility is not limited to the sequences listed below:

Numerous SNPs are reported to be associated with hair loss (alopecia). Table 1 lists candidate SNPs and/or genes in which the SNP occurs.

TABLE 1
Skin and hair disorders

| SNP ID | Gene | Disease |
|---|---|---|
| rs6152 | AR | Alopecia |
| Rs2223841 | AR | Alopecia |
| Rs1160312 | PAX1, FOXA2 | Alopecia |
| Rs2180439 | HDAC9 | Alopecia |
| Rs12565727 | TARDBP | Alopecia |
| Rs9287638 | HDAC4 | Alopecia |
| Rs2073963 | HDAC9 | Alopecia |
| Rs6945541 | AUTS2 | Alopecia |
| Rs12373124 | 17q21.31 | Alopecia |
| Rs10502861 | SETBP1 | Alopecia |
| Rs6047844 | PAX1, FOXA2 | Alopecia |
| Rs2497938 | AR | Alopecia |
| Rs187238 | IL-18 | Alopecia |
| Rs1946518 | IL-18 | Alopecia |
| Rs6010620 | TNFRSF6B, ZGPAT | Atopic dermatitis |

TABLE 2
SNPs associated with Neurological Disorders

| SNP ID | Gene | Disease |
|---|---|---|
| Rs237887 | Oxytocin Receptor | Autism Spectrum |
| Rs2268491 | Oxytocin Receptor | Autism Spectrum |
| Rs2254298 | Oxytocin Receptor | Autism Spectrum |
| Rs7632287 | Oxytocin Receptor | Autism Spectrum |
| rs17525809 | P2RX7 | Multiple Sclerosis |
| Rs28360447 | P2RX7 | Multiple Sclerosis |
| Rs28360447 | P2RX7 | Chronic Pain |
| Rs7958311 | P2RX7 | Chronic Pam |
| Rs1718119 | P2RX7 | Anxiety disorder |
| Rs2230912 | P2RX7 | Bipolar disorder, depression |
| Rs8028149 | lncRNA | Major Depression |

Systemic diseases may require delivery of the invention via vascular routes (intra-venous, intra-arterial). Table 3 identifies examples of SNPs associated with diseases that may be best treated through systemic delivery routes.

TABLE 3
Other SNPs and associated Diseases (Orthopedic,
Autoimmune, general metabolic diseases etc)

| SNP ID | Gene | Disease |
|---|---|---|
| Rs28360447 | P2X7 | Osteoporosis |
| Rs28360457 | P2X7 | Osteoporosis |
| Rs1718119 | P2RX7 | Osteoporosis |
| Rs2230911 | P2RX7 | Osteoporosis |
| Rs1653624 | P2RX7 | Osteoporosis |
| Rs11669309 | NCRNA | Hypertension |
| Rs2269706 | HLA gene | Rheumatoid arthritis |
| rs16868911, rs11543230, | lncRNA | Multiple Complex |
| rs9309325, rs12951337 | | Diseases |
| rs15428265 | lncRNA | Celiac disease |
| rs12683158 | lncRNA | Type II diabetes |
| rs6982502 | lncRNA | Coronary Heart |
| | | Disease |
| Rs11669309 | ncRNA | Hypertension |

For example, in one aspect disclosed herein are methods of treating, inhibiting, reducing, ameliorating, preventing, and/or reversing a disease or disorder, wherein the disease or disorder is baldness (such as, for example, male pattern baldness, androgenetic alopecia, alopecia areata, cicatricial alopecia, telogen effluvium, or female pattern baldness), and wherein single nucleotide polymorphism (SNP) associated with the baldness in the subject comprises one or more of the SNPs Rs6152, Rs 2223841, Rs2497938, Rs1160312, 6047844, Rs2180439, Rs2073963, Rs12565727, Rs9287638, Rs6945541, Rs12373124, Rs10502861, Rs187238, and/or Rs1946518 or the SNP is associated with the AR, PAX1, FOXA2, TARDBP, HDAC4, HDAC9, AUTS2, 17₈21.31, SETBP1, IL18, Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

In one aspect, disclosed herein are methods of treating, inhibiting, reducing, ameliorating, preventing, and/or reversing a disease or disorder, wherein the disease or disorder comprises autism spectrum disorder and the SNP associated with the autism spectrum disorder comprise one or more of the SNPs Rs237887, Rs 2268491, Rs2254298, and/or Rs 7632287.

Also disclosed herein are methods of treating, inhibiting, reducing, ameliorating, preventing, and/or reversing a disease or disorder, wherein the disease or disorder comprises osteoporosis and the SNP associated with the osteoporosis comprises one or more of the SNPs Rs28360447, Rs28360457, Rs1718119, Rs2230911, and/or Rs1653624.

In one aspect, disclosed herein are methods of treating, inhibiting, reducing, ameliorating, preventing, and/or reversing a disease or disorder, wherein the disease or disorder comprises Rheumatoid Arthritis and the SNP associated with the Rheumatoid Arthritis comprises the SNP Rs2269706.

Also disclosed herein are methods of treating, inhibiting, reducing, ameliorating, preventing, and/or reversing a disease or disorder, wherein the disease or disorder comprises Multiple Sclerosis and the SNP associated with the Multiple Sclerosis comprises one or more of the SNPs Rs17525809 and/or Rs28360447.

In one aspect, disclosed herein are methods of treating, inhibiting, reducing, ameliorating, preventing, and/or reversing a disease or disorder, wherein the disease or disorder comprises chronic pain and the SNP associated with the chronic pain comprises one or more of the SNPs Rs28360447 and/or Rs7958311.

Also disclosed herein are methods of treating, inhibiting, reducing, ameliorating, preventing, and/or reversing a disease or disorder, wherein the disease or disorder comprises atopic dermatitis and the SNP associated with the atopic dermatitis comprises the SNP Rs6010620.

In one aspect, disclosed herein are methods of treating, inhibiting, reducing, ameliorating, preventing, and/or reversing a disease or disorder, wherein the disease or disorder comprises hypertension and the SNP associated with the hypertension comprises the SNP Rs11669309.

Also disclosed herein are methods of treating, inhibiting, reducing, ameliorating, preventing, and/or reversing a disease or disorder, wherein the disease or disorder comprises coronary heart disease and the SNP associated with the heart disease comprises the SNP Rs6982502.

In one aspect, disclosed herein are methods of treating, inhibiting, reducing, ameliorating, preventing, and/or reversing a disease or disorder, wherein the disease or disorder comprises type II diabetes and the SNP associated with the type II diabetes comprises the SNP Rs12683158.

Also disclosed herein are methods of treating, inhibiting, reducing, ameliorating, preventing, and/or reversing a disease or disorder, wherein the disease or disorder comprises celiac disease and the SNP associated with the celiac disease comprises the SNP Rs15428265.

In one aspect, disclosed herein are methods of treating, inhibiting, reducing, ameliorating, preventing, and/or reversing a disease or disorder, wherein the disease or disorder anxiety and the SNP associated with the anxiety comprises the SNP Rs1718119.

Also disclosed herein are methods of treating, inhibiting, reducing, ameliorating, preventing, and/or reversing a disease or disorder, wherein the disease or disorder comprises bipolar disorder and the SNP associated with the bipolar disorder comprises the SNP Rs2230912.

In one aspect, disclosed herein are methods of treating, inhibiting, reducing, ameliorating, preventing, and/or reversing a disease or disorder, wherein the disease or disorder comprises depression and the SNP associated with the depression comprises the SNP Rs8028149.

Also disclosed herein are methods of treating, inhibiting, reducing, ameliorating, preventing, and/or reversing a disease or disorder of any preceding aspect, wherein the disease or disorder comprises the one or more of the SNPs Rs16868911, Rs11543230, Rs9309325, and/or Rs12951337.

As noted above, the disclosed treatment can be beneficial to treat any disease where uncontrolled cellular proliferation occurs such as cancers. Thus, also disclosed herein are methods of treating, inhibiting, reducing, ameliorating, preventing, and/or reversing a disease or disorder, wherein the disease or disorder is a cancer. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Accordingly, in one aspect, disclosed herein are methods of treating, inhibiting, reducing, ameliorating, preventing, and/or reversing a cancer, wherein the cancer comprises pancreatic cancer or breast cancer. In one aspect, disclosed herein are methods of treating, inhibiting, reducing, ameliorating, preventing, and/or reversing a disease or disorder, wherein the disease is breast cancer and the SNP associated with the breast cancer in the subject comprises the SNP Rs2298075. Also disclosed herein are methods of treating, inhibiting, reducing, ameliorating, preventing, and/or reversing a disease or disorder of any preceding aspect, wherein the disease is pancreatic cancer and the SNP associated with the pancreatic cancer in the subject comprises the SNP Rs10237038.

Baldness

Hair is made of a protein called keratin. Hair sits in a hair follicle, and at the base of the follicle are stem cells, called follicular epithelial stem cells, that stimulated the production of new hair. The average head has over 100,000 hair follicle and each follicle cycles producing hair and then resting. The cycle of producing hair and resting is normal and results in always having a full head of hair.

Baldness occurs because more and more hair follicles are in the resting stage or shrink until they never produce new hair. Because of genetics, men destined to be bald have hair follicles that are overly sensitive to the actions of dihydrotestosterone (DHT), which is a byproduct of testosterone. DHT binds to hair follicles and causes them to shrink. More and more hair follicles shrink until they product no hair, which causes areas to develop hair thinning and eventual baldness. This process is called miniaturization.

Currently, only two drugs are approved by the FDA to treat hair loss—minoxidil and finasteride. Minoxidil is available as an over-the-counter topical, but most experts agree that minoxidil is a relatively marginal effective drug in the fight against hair loss and has zero effect on the process of miniaturization. Thus, its benefits are temporary. Finasteride works by inhibiting testosterone become DHT, but its side effects include erectile dysfunction and libido and ejaculation disorders. Low level laser therapy has been shown to stimulate hair growth, in both men and women, but has no effect on miniaturization.

Hair transplantation is another method for treating baldness and involves harvesting follicles from the back of the head that are DHT resistant and transplanting them to bald areas. However, this does not stimulate new growth as the patient still has the same amount of hair, it is just redistributed more evenly around the scalp. What is needed is a composition and method for preventing and treating baldness, wherein the method and composition promotes the stimulation of the follicular epithelial stem cells and mesenchymal stem cells in the follicle to result in activated follicles to produce hair and prevent miniaturization.

Some embodiments of the present disclosure include a method for producing a composition for preventing and treating baldness comprising preparing a concentration of mesenchymal stem cell (MSC) exosomes and secretomes from a targeted donor with specific SNP hair characteristics that indicate the donor will never experience hair loss. The donor may be gender specific. The method may include identifying the target donor by SNP hair characteristics, culturing MSCs from the target donor to create a cultured media under either normal hyperoxic culturing conditions or harsh wound healing hypoxic conditions; creating a powder from the cultured media; and combining the powder with a cream or lotion base, wherein the final product may be applied to the dermis in areas affected by miniaturization. Application may be either topical or injectable and may stimulate the activation of hair follicles to promote hair growth, particularly in areas affected by miniaturization or by direct injection into the dermis.

When injected into the scalp of the affected areas of subjects with male pattern baldness or androgenetic alopecia, successful hair regrowth was observed in as little as 2 weeks post treatment (see FIGS. 2A-2E).

A proteomic assessment of exosome suspensions generated using methods and parameters described herein was performed to characterize the molecular compositions that contribute to clinically relevant efficacy of the described invention. The proteomic assessments utilized commercially available antibody arrays manufactured by RayBio Tech, Norcross, Ga., USA). Concentration measurements were made of 230 different proteins known to either be secreted, transported or present on the external surface of cell membranes (within the extracellular microenvironment). Proteins measured present at physiologically relevant concentrations in duplicate test samples and which were found to be supported as relevant to hair restoration and colorization are listed in Table 4. A physiologically relevant concentration was deemed to be a mean concentration of ≥1 pg/mL. Literature surveys of proteins with average concentrations greater than 50 ug/mL were performed to identify most likely candidate molecules to support a clinical effect. The list in Table 4 should Not be considered to be a comprehensive or exhaustive list of individual proteins found within the invention that are relevant to efficacious effects.

TABLE 4
Survey of Invention Protein Content.

| | Mean | Exosome | Exosome |
|---|---|---|---|
| Protein | Concentration | Suspension | Suspension |
| Identity | [pg/mL] | Sample 1 [pg/mL] | Sample 2 [pg/mL] |
| IL-18 | 50033.50 | 29801.41 | 70265.59 |
| PDGF Rb | 10178.43 | 10036.71 | 10320.16 |
| IGFBP-4 | 5840.14 | 3878.93 | 7801.34 |
| TIMP-2 | 1708.44 | 2121.46 | 1295.42 |
| TIMP-1 | 684.26 | 734.23 | 634.29 |
| IL-23 | 372.10 | 393.96 | 350.24 |
| Activin A | 454.81 | 378.14 | 531.48 |
| ICAM-2 | 193.61 | 365.61 | 21.60 |
| XEDAR | 226.68 | 317.96 | 135.40 |
| OPN | 254.12 | 252.85 | 255.38 |
| TNF RI | 126.27 | 136.16 | 116.37 |
| Follistatin | 95.93 | 129.93 | 61.93 |

Proteins are organized from highest to lowest mean concentration. Proteins expressed at concentrations greater than the arbitrary value of 50 ng/mL were surveyed in the medical literature using PubMed search engine to identify studies that provide evidence for potential effects of this invention. Proteins in the invention supported in the scientific literature to play a role in hair follicle regeneration are listed and described below.

IL18 was the most concentrated protein present in the exosome suspension samples. It's primary function involves regulation of the innate immune response within the skin, specifically, stimulating interferon gamma production and activating dermal natural killer and TH1 T-cells. Too much IL18 is associated with onset autoimmune diseases: The literature survey provides evidence for a role in hair growth. Two single nucleotide polymorphisms (SNPs) found within IL18 gene sequence are associated with development of the organ specific autoimmune disease, alopecia areata (AA). Two specific SNPs, rs1946518 (−607C>A) and rs187238 (−137G>C) polymorphisms are associated with alopecia areata disease. Celik et al concluded that IL-18 rs187238 and rs1946518 SNPs may be the cause of the AA susceptibility.¹ IL18 may play a role in observed irregular interactions between perifollicular mast cells and CD8+ cells. These interactions may disrupt the normal hair growth cycle within the follicles. By histology analysis, IL18 and its receptor are found within skin keratinocytes, and within the outer sheath cells of hair follicles. Two possible effects of IL18 action to initiate hair growth observed when the invention is applied to the dermis is that the sequence of the IL18 from this donor does not have the disease associated sequences and changes IL18 signaling levels to a non-disease state level. Alternatively, evidence of IL18 isoforms exists including a predominant form in sera that may function as an autocrine inhibitor. The form of IL18 in this invention may be an inhibitory isoform that reduces IL18 signaling and restores an appropriate the innate immune response to an appropriate level.

It has been demonstrated that induction of anagen phase using conditioned media from hypoxic adipose derived mesenchymal stem/stromal cell populations. PDGF-receptor B was a major protein detected and. PDGF may stimulate dermal papillae proliferation through the PDGF receptor. Exosomic introduction of PDGF-Receptor B into membranes of telogenic dermal papillae cells may enable signaling via PDGF that initiates anagenic hair growth. Genetic variants of PDGFR-B are associated with hair loss in Penttinen Syndrome.

Insulin growth factors are agonists for hair growth. IGF binding proteins regulate IGF activities by binding to IGF. Binding to the various IGF-BPs can modify IGF activity and provide additional specificity of IGF activity. IGF-BP4, along with IGF-BP3 and IGF-BP5 are expressed inhuman hair follicle dermal papillae and serve to regulate IGF activity within the hair follicle.

Normal hair growth is cyclic. Each hair follicle undergoes extracellular remodeling throughout each cycle. TIMP-1 and TIMP-2 play critical roles in regulating proteolytic activity of collagenases and other proteases involved remodeling of extracellular matrix in and around the hair follicle. Additional TIMPs provided by the invention may restore balance to levels of proteases altered by inflammatory disease states associated with alopecia.

Interleukin twenty-three is found in hair follicles at a higher level during Alopecia Areata; however, it's exact function within the hair follicles is still unknown. Like other cytokines, IL23 may have multiple functions depending on isoform, and receptors expressed by different cell types. Additional IL23 provided by the invention could provide an autocrine inhibitory signal that helps decrease the inflammatory condition resulting in hair growth suppression

The activins are members of the TGF-B signaling pathway and are critical for the initial formation of hair follicles during development, and they play a key role in epidermal/mesenchymal interactions required during hair organogenesis. Subsequently, in combination with follistatin, activin is an important regulator of the hair cycle.

ICAMs function to connect and create a barrier between cells. The hair follicle is an immune-privileged micro-organ. ICAMs establish physical barriers that establish that immune privilege environment. In alopecia areata, the immune privileged environment is disrupted enabling development of an autoimmune response to antigens within the melanocytes that provide color to the hair. By providing ICAM-2, the invention may enable reestablishment of the immune privileged micro-environment

Osteopontin is expressed by outer hair follicle sheath cells. Osteopontin is proteolytically cleaved in vivo to generate peptide signaling molecules that regulate FGF-7 production by the outer root sheath keratinocytes. The osteopontin derived peptide appears to inhibit FGF-7 synthesis which in turns slows hair growth. Osteopontin therefore may be an important regulator of the hair growth cycle.

EDAR and XEDAR bind ectodysplasin family members Eda A1 and Eda A2. XEDAR activates NFKB signaling path and is associated with signaling during hair follicle morphogenesis. Murine gene knockout studies of this signaling pathway leads to malformation of hair follicles.

### Mesenchymal Stem Cells

As noted throughout, the treatment compositions disclosed herein can utilize exosomes and/or growth factors derived from mesenchymal stem cells (MSCs). While existing autogenous and allogeneic MSCs contained within bone marrow concentrate or adipose-derived stromal vascular fraction (SVF) or various post-natal products from umbilical cord, placenta or amnion, expanded MSC cultures are currently being used to treat wounds, orthopedic pathology, and spine pathology; the existing treatments do not contain large amounts of MSC secretomes (including, but not limited to growth factors, cytokines, chemokines, exosomes, extracellular vesicles, and/or extracts). Additionally, despite evidence in the art that treatments comprising stem cells (including injectable treatments) can help prevent aging and treat scarring, uneven pigmentation, existing skin products, such as creams, lotions, serums, make-up, and the like, while including ingredients that potentially help treat and strengthen the skin, other topical products do not penetrate the epidermis and more importantly do not include human MSCs, or MSC-derived growth factors and proteins. In fact, prior to the present disclosure an active MSC growth factor product that can be used for these applications has not been developed. Thus, in one aspect, disclosed herein are MSC secretome compositions (including, but not limited to MSC growth factor, MSC exosome, MSC extracts and/or extracellular vesicle comprising compositions) for use in the treatment of wounds, orthopedic disorders, orthopedic injuries, ophthalmology, spinal injury, or spinal disorders, said treatment compositions comprising (i) a growth factor powdered additive comprising a mesenchymal stem cell (MSC) derived preparation and (ii) a pharmaceutically acceptable carrier.

As noted above, MSC are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “MSC preparation” or “MSC secretome composition” refers to a composition comprising MSC growth factors, MSC exosomes, extracellular vesicles, or acellular extracts of MSCs or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract, MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxyic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. As used herein, MSC growth factors include but are not limited to prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

In one aspect, the MSC preparation (such as, for example, a MSC secretome composition) comprises MSC growth factors, MSC exosomes, and/or cellular extracts of MSCs or MSC lysates obtained from MSCs cultured under standard hyperoxyic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0-7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

The combined reduced nutrient and metabolite environment (artificial wound healing) can trigger the cultured cells to produce wound healing and anti-inflammatory ECM proteins and growth factors and extracellular vesicles that are there to direct tissue healing, which can be in the form of new ECM proteins, such as collagen and glycosaminoglycans (GAGs) as well as growth factors and cytokines. Thus, it is understood and herein contemplated that by adjusting the cell growth conditions, such as cell confluency, culture media supplements, nutritional supplements, oxygen levels, length of culture in those conditions, cell passage number or combinations of those, and the like, MSCs can be stimulated to selectively secrete the desired anti-inflammatory proteins, peptides, cytokines, chemokines, glycosaminoglycans, extracellular matrix (ECM), proteoglycans, exosomes and secretomes.

In one aspect, it is understood and herein contemplated that the growth conditions such as temperature, oxygen tension, pH, glucose saturation, confluency, and growth surface can affect the gene expression and protein production of cells growing in culture and thereby can result in different growth factors and cytokines being produced. For example, growth surface stiffness (Young's Modulous) affects the gene expression and protein production of the cells growing on it. Adipose cells and cartilage cells are usually maintained on a softer and more elastic growth surface (˜10 kPa-12 kPa), while bone cells are better grown on a stiff surface (˜10⁶-12⁶ kPa). By adjusting the surface stiffness, it is possible to influence the secretomes of the cells and their communication signals (growth factors, exosomes, cytokines and chemokines).

In one aspect, the MSC secretome compositions (including, but not limited to MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the protective coating can comprise a biocompatible polymer. In one aspect, biocompatible polymer can be crosslinked. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly (isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof, Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non-reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, α-D-Glucopyranosyl-D-glucose, α-D-Glucopyranosyl-dihydrate, Polymer of β-D-Glycopyranosyl units, β-D-Fructofuranosyl α-D-glucopyranoside (anhydrous/dihydrate), β-D-Galactopyranosyl-D-glucose, α-D-Glucopyranosyl-α-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-β-(1-4)-anhydro-L-galactosyl, Cellulose, Polymer of β-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be pnat or animal sources.

In one aspect, any of the polymers, monosaccharides, disaccharides, or polysaccharides used to form the protective coating formed by placing the MSC additive in a encapsulating solution can be at an appropriate concentration for form the protective coating. For example, polymers, monosaccharides, disaccharides, or polysaccharides can be at any concentration between 0.01 mM and 10.0M concentration, for example, from about 0.01M to about 0.1M, from about 0.1 mM to about 1.0M, from about 1.0M to about 10.0M. Exemplary concentrations include 0.01, 0.02, 0.03, 0.04, 0.05, 0.06, 0.07, 0.08, 0.09, 0.1, 0.2, 0.3, 0.4, 0.4, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 225, 250, 275, 300, 325, 350, 375, 400, 450, 500, 600, 700, 800, 900 mM, 1, 1.1, 1.2, 1.3, 1.4, 1.5, 1.6, 1.7, 1.8, 1.9, 2, 3, 4, 5, 6, 7, 8, 9, 10M.

As shown in FIGS. 1 and 2, the exosomses and extracellular vesicles in the disclosed MSC secretome compositions have been produced.

In one aspect, it is understood and herein contemplated that one way to treat a wound is through administration of the MSC secretome compositions (including, but not limited to MSC growth factor, MSC exosome, MSC extracts and/or extracellular vesicle comprising compositions) subcutaneously, intramuscularly, intravenously, topically (such as, for example, through the use of salves, creams, and/or ointments), but also by impregnating stents, sponges, matrixes, scaffolds, bandages, dressing, sutures, grafts, surgical drapes, surgical adhesive, and/or staples with the MSC secretome compositions. Thus, in one aspect, disclosed herein are medicated stents, scaffolds, sponges, matrixes, adhesive bandages, wound dressings, grafts, surgical drapes, sutures, salves, creams, or wound adhesives comprising a therapeutically effective amount of the MSC secretome composition. The MSC secretome compositions (including, but not limited to MSC growth factor, MSC exosome, MSC extracts and/or extracellular vesicle comprising compositions), as noted above, can be administered topically and applied to the face, the neck, the hands, or any other desired part of the body. When applied to an adhesive bandage, wound dressing, grafts, surgical drape, suture, scaffold, matrix, sponge, or stent, the MSC secretome composition can be a applied as a powder.

In one aspect, the MSC secretome compositions (including, but not limited to MSC growth factor, MSC exosome, MSC extracts and/or extracellular vesicle comprising compositions) disclosed herein may comprise any known ingredients typically found in the wound healing fields, such as oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as Ω-3, Ω-6, and Ω-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

It is understood and herein contemplated that the MSC secretome compositions disclosed herein can provide wound healing rejuvenation, augmentation, and improved or restored skin tissue. The composition may also be used as an injectable in the treatment of joint arthritis and degenerated spinal discs. Moreover, embodiments of the composition may not require the inclusion of additional growth factors or hormones, such as insulin, insulin-like growth factors, thyroid hormones, fibroblast growth factors, estrogen, retinoic acid, and the like. In some aspect, the disclosed stem cell growth factor compositions can comprise additional active ingredients including, but not limited to antibiotics, anti-acne agents, liposomes, antioxidants, platelet-rich fibrin matrixes, analgesic, anti-inflammatories, as well as, additional growth factors, such as insulin, insulin-like growth factors, thyroid hormones, fibroblast growth factors, estrogen, retinoic acid, and the like. Such additional active ingredients can be mixed with the stem cell growth factor and extracellular vesicle compositions disclosed herein as well as MSC conditioned media, MSC lystates, and MSC-derived produces and then thawed or dissolved, mixed, or suspended in a mixture of emulsifying lanolin alcohols, waxes, and oils or a mixture of petrolatum or mineral oil, a quaternary ammonium compound, a fatty alcohol, and a fatty ester emollient, or lotions that are substantially similar in composition.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. I Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The preceding examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### C. REFERENCES

Alam M, Bertolini M, Gherardini J, Keren A, Ponce L, Chéret J, Alenfall J, Dunér P, Nilsson A H, Gilhar A, Paus R. An osteopontin-derived peptide inhibits human hair growth at least in part by decreasing fibroblast growth factor-7 production in outer root sheath keratinocytes. Br J Dermatol. 2019
Aversa A, Basciani S, Visca P, Arizzi M, Gnessi L, Frajese G, Fabbri A. Platelet-derived growth factor (PDGF) and PDGF receptors in rat corpus cavernosum: changes in expression after transient in vivo hypoxia. J Endocrinol. 2001 August; 170(2):395-402.
Batch J A, Mercuri F A, Werther G A. Identification and localization of insulin-like growth factor-binding protein (IGFBP) messenger RNAs in human hair follicle dermal papilla. J Invest Dermatol. 1996 March; 106(3):471-5.
Bertolini M, Zilio F, Rossi A, et al. Abnormal interactions between perifollicular mast cells and CD8+ T-cells may contribute to the pathogenesis of alopecia areata. PLoS ONE. 2014; 9:e94260.
Boraschi D, Dinarello C A. IL-18 in autoimmunity: review. Eur Cytokine Netw. 2006; 17:224-Fouad N A, Baraka E A, Hassan W A. Interleukin-18 gene polymorphisms in systemic lupus erythematosus: relation to disease status. Egypt J Immunol. 2014; 21:1-12.
Botchkarev V A, Fessing M Y. Edar signaling in the control of hair follicle development. J Investig Dermatol Symp Proc. 2005 December; 10(3):247-51.
Celik S D, Ates O. Genetic analysis of interleukin 18 gene polymorphisms in alopecia areata. J Clin Lab Anal. 2018 June; 32(5):e22386.
Chen C C, Murray P J, Jiang T X, Plikus M V, Chang Y T, Lee O K, Widelitz R B, Chuong C M. Regenerative hair waves in aging mice and extra-follicular modulators follistatin, dkk1, and sfrp4. J Invest Dermatol. 2014 August; 134(8):2086-2096.
Chun Hou, Yong Miao, Xue Wang, Chaoyue Chen, Bojie Lin, Zhiqi Hu. Expression of matrix metalloproteinases and tissue inhibitor of matrix metalloproteinases in the hair cycle Exp Ther Med. 2016 July; 12(1): 231-237. Published online 2016 May 9.
Gilhar A. Collapse of immune privilege in alopecia areata: coincidental or substantial? J Invest Dermatol. 2010 November; 130(11):2535-7.
Kawabe T T, Rea T J, Flenniken A M, Williams B R, Groppi V E, Buhl A E. Localization of TIMP in cycling mouse hair. Development. 1991 April; 111(4):877-9
Kim S K, Park H J, Chung J H, et al. Association between interleukin 18 polymorphisms and alopecia areata in Koreans. J Interferon Cytokine Res. 2014; 34:349-353.
McDowall M, Edwards N M, Jahoda C A, Hynd P I. The role of activins and follistatins in skin and hair follicle development and function. Cytokine Growth Factor Rev. 2008 October December; 19(5-6):415-26.
Park B S, Kim W S, Choi J S, Kim H K, Won R I, Ohkubo F. Fukuoka H. Hair growth stimulated by conditioned medium of adipose-derived stem cells is enhanced by hypoxia: evidence of increased growth factor secretion. Biomed Res. 2010 February; 31(1):27-34
Sivalingam S P, Yoon K H, Koh D R, Fong K Y. Single-nucleotide polymorphisms of the interleukin-18 gene promoter region in rheumatoid arthritis patients: protective effect of AA genotype. Tissue Antigens. 2003; 62:498-504.
Suárez-Fariñas M, Ungar B, Noda S, Shroff A, Mansouri Y, Fuentes-Duculan J, Czemik A, Zheng X, Estrada Y D, Xu H, Peng X, Shemer A, Krueger J G, Lebwohl M G, Guttman-Yassky E. Alopecia areata profiling shows TH1, TH2, and IL-23 cytokine activation without parallel TH17/TH22 skewing. J Allergy Clin Immunol. 2015 November; 136(5):1277-87.
Wang G, Dai D, Chen X, Yuan L, Zhang A, Lu Y, Zhang P. Upregulation of neuregulin-1 reverses signs of neuropathic pain in rats. Int J Clin Exp Pathol. 2014 Aug. 15; 7(9):5916-21.
Wang X, Chen H, Tian R, Zhang Y, Drutskaya M S, Wang C, Ge J, Fan Z, Kong D, Wang X, Cai T, Zhou Y, Wang J, Wang J, Wang S, Qin Z, Jia H, Wu Y, Liu J, Nedospasov S A, Tredget E E, Lin M, Liu J, Jiang Y, Wu Y. Macrophages induce AKT/β-catenin-dependent Lgr5(+) stem cell activation and hair follicle regeneration through TNF. Nat Commun. 2017 Mar. 27; 8:14091.

What is claimed is:

1. A method for creating mesenchymal stem cell and exosome treatment composition for treating, inhibiting, reducing, ameliorating, preventing, and/or reversing a disease or disorder in a subject, the method comprising:
a) identifying the hair, skin color, skin type, race, ethnicity of an end user;
b) identifying a single nucleotide polymorphism (SNP) associated with the disease or disorder in the subject;
c) obtaining mesenchymal stem cells (MSCs) from a targeted donor having the same hair type, color, and/or ethnicity as the subject but with a single nucleotide polymorphism (SNP) profile that indicates the donor will never experience disease or disorder; and
d) preparing an MSC and exosome preparation from the obtained MSCs, wherein the MSC and exosome preparation is created by culturing MSCs in media comprising growth conditions sufficient to generate MSCs and exosomes that comprise and or secrete corrective SNPs and cytokines.

2. The method of claim 1, wherein the method further comprises identifying the type and amount of growth factors in the MSC and exosome preparation.

3. The method of claim 2, wherein growth factors are identified through proteomic analysis.

4. The method of claim 1, wherein the method further comprises characterizing the exosome RNA.

5. The method of claim 4, wherein the exosome RNA is obtained by sequencing.

6. The method of claim 1, wherein the disease or disorder comprises baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer.

7. The method of claim 6, wherein the baldness comprises male pattern baldness, androgenetic alopecia, alopecia areata, cicatricial alopecia, telogen effluvium, or female pattern baldness.

8. The method of claim 7, wherein the treatment composition stimulates the activation of hair follicles to promote hair growth.

9. The method of claim 6, wherein the cancer comprises pancreatic cancer or breast cancer.

10. The method of claim 1, wherein the SNP comprises one or more SNPs present in one or more genes encoding Interleukin 18 (IL-18), Androgen Receptor (AR), Histone deacetylase (HDAC)-4 (HDAC4), HDAC9, Paired Box 1 (PAX1), Forkhead box A2 (FOXA2), TAR DNA Binding Protein (TARDBP), Autism Susceptibility Gene 2 (AUTS2), SET Binding Protein 1 (SETBP1), 17q21.31, tumor necrosis factor receptor superfamily member 6B (TNFRSF6B), Zinc finger CCCH-type with G patch domain-containing protein (ZGPAT), P2X purinoceptor 7 (P2RX7), Oxytocin Receptor, non-coding RNA (ncRNA), long non-coding RNA (lncRNA), human leukocyte antigen (HLA), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

11. The method of claim 10, wherein the SNP comprises the IL-18 SNP rs1946518 and/or rs187238.

12. The method of claim 10, wherein the SNP comprises ncRNA SNP Rs11669309, Rs2298075, and/or Rs10237038.

13. The method of claim 10, wherein the SNP comprises the androgen receptor SNP Rs6152, Rs 2223841, and/or Rs2497938.

14. The method of claim 10, wherein the SNP comprises the oxytocin receptor SNP Rs237887, Rs 2268491, Rs2254298, and/or Rs 7632287.

15. The method of claim 10, wherein the SNP comprises the P2RX7 SNP Rs17525809, Rs28360447, Rs7958311, Rs1718119, Rs2230912, Rs28360457, Rs2230911, and/or Rs1653624.

16. The method of claim 10, wherein the SNP comprises the lncRNA SNP Rs8028149, Rs16868911, Rs11543230, Rs9309325, Rs12951337, Rs15428265, Rs12683158, and/or Rs6982502.

17. The method of claim 10, wherein the SNP comprises the HLA gene SNP Rs2269706.

18. The method of claim 10, wherein the SNP comprises the PAX1/FOXA2 SNP Rs1160312 and/or Rs6047844.

19. The method of claim 10, wherein the SNP comprises the HDAC9 SNP Rs2073963 and/or Rs2180439.

20. The method of claim 10, wherein the SNP comprises the HDAC4 SNP Rs9287638.

21. The method of claim 10, wherein the SNP comprises the TARDBP SNP Rs12565727.

22. The method of claim 10, wherein the SNP comprises the AUTS2 SNP Rs6945541.

23. The method of claim 10, wherein the SNP comprises the 17q21.31 SNP Rs12373124.

24. The method of claim 10, wherein the SNP comprises the SETBP1 SNP Rs10502861.

25. The method of claim 10, wherein the SNP comprises the TNFRSF6B/ZGPAT SNP Rs6010620.

26. A mesenchymal stem cell and exosome treatment composition for use in treating a disease or disorder in an end user subject, the composition comprising:
a) a composition base; and
b) a mesenchymal stem cell and exosome preparation derived from a donor having the same hair type, hair color, skin type, skin color, race, and/or ethnicity as an end user but with a single nucleotide polymorphism (SNP) profile that indicates the donor will never disease or disorder suffered by the end user subject; wherein the MSC and exosome preparation comprises at least one member selected from the group consisting of cells or cell conditioned media cultured under normal hyperoxic culturing conditions and cells cultured under harsh wound healing conditions.

27. The mesenchymal stem cell and exosome treatment composition of claim 26, wherein the SNP comprises one or more SNPs present in one or more genes encoding Interleukin 18 (IL-18), Androgen Receptor (AR), Histone deacetylase (HDAC)-4 (HDAC4), HDAC9, Paired Box 1 (PAX1), Forkhead box A2 (FOXA2), TAR DNA Binding Protein (TARDBP), Autism Susceptibility Gene 2 (AUTS2), SET Binding Protein 1 (SETBP1), 17q21.31, tumor necrosis factor receptor superfamily member 6B (TNFRSF6B), Zinc finger CCCH-type with G patch domain-containing protein (ZGPAT), P2X purinoceptor 7 (P2RX7), Oxytocin Receptor, non-coding RNA (ncRNA), long non-coding RNA (lncRNA), human leukocyte antigen (HLA), Platelet Derived Growth Factor (PDGF) receptor (PDGFR) B (PDGFR-B), Tissue Inhibitors of Metalloprotease (TIMP) 1 (TIMP-1), TIMP-2, IL-23, Activin A, intrcellular adhesion molecule (ICAM-2), Osteopontin (OPN), Insulin, Insulin growth factor binding protein four (IGF-BP4), Tumor Necrosis Factor (TNF) receptor 1 (TNF-R1), Ectodysplasin A2 receptor (XEDAR), and/or follistatin.

28. The mesenchymal stem cell and exosome treatment composition of claim 27, wherein the SNP comprises the IL-18 SNP rs1946518 and/or rs187238.

29. The mesenchymal stem cell and exosome treatment composition of claim 27, wherein the SNP comprises ncRNA SNP Rs11669309, Rs2298075, and/or Rs10237038.

30. The mesenchymal stem cell and exosome treatment composition of claim 27, wherein the SNP comprises the androgen receptor SNP Rs6152, Rs 2223841, and/or Rs2497938.

31. The mesenchymal stem cell and exosome treatment composition of claim 27, wherein the SNP comprises the oxytocin receptor SNP Rs237887, Rs 2268491, Rs2254298, and/or Rs 7632287.

32. The mesenchymal stem cell and exosome treatment composition of claim 27, wherein the SNP comprises the P2RX7 SNP Rs17525809, Rs28360447, Rs7958311, Rs1718119, Rs2230912, Rs28360457, Rs2230911, and/or Rs1653624.

33. The mesenchymal stem cell and exosome treatment composition of claim 27, wherein the SNP comprises the lncRNA SNP Rs8028149, Rs16868911, Rs11543230, Rs9309325, Rs12951337, Rs15428265, Rs12683158, and/or Rs6982502.

34. The mesenchymal stem cell and exosome treatment composition of claim 27, wherein the SNP comprises the HLA gene SNP Rs2269706.

35. The mesenchymal stem cell and exosome treatment composition of claim 27, wherein the SNP comprises the PAX1/FOXA2 SNP Rs1160312 and/or Rs6047844.

36. The mesenchymal stem cell and exosome treatment composition of claim 27, wherein the SNP comprises the HDAC9 SNP Rs2073963 and/or Rs2180439.

37. The mesenchymal stem cell and exosome treatment composition of claim 27, wherein the SNP comprises the HDAC4 SNP Rs9287638.

38. The mesenchymal stem cell and exosome treatment composition of claim 27, wherein the SNP comprises the TARDBP SNP Rs12565727.

39. The mesenchymal stem cell and exosome treatment composition of claim 27, wherein the SNP comprises the AUTS2 SNP Rs6945541.

40. The mesenchymal stem cell and exosome treatment composition of claim 27, wherein the SNP comprises the 17q21.31 SNP Rs12373124.

41. The mesenchymal stem cell and exosome treatment composition of claim 27, wherein the SNP comprises the SETBP1 SNP Rs10502861.

42. The mesenchymal stem cell and exosome treatment composition of claim 27, wherein the SNP comprises the TNFRSF6B/ZGPAT SNP Rs6010620.

43. A method of treating, inhibiting, reducing, ameliorating, preventing, and/or reversing a disease or disorder comprising administering to a subject any of the mesenchymal stem cell and exosome treatment composition of any of claims 26-42.

44. The method of claim 43, wherein the disease or disorder comprises baldness, atopic dermatitis, Autism spectrum disorder, Multiple sclerosis, chronic pain, depression, anxiety, bipolar disorder, osteoporosis, rheumatoid arthritis, celiac disease, type II diabetes, coronary heart disease, hypertension, multiple complex diseases, and/or cancer.

45. The method of claim 44, wherein the baldness comprises male pattern baldness, androgenetic alopecia, alopecia areata, cicatricial alopecia, telogen effluvium, or female pattern baldness.

46. The method of claim 45, wherein the single nucleotide polymorphism (SNP) associated with the disease or disorder in the subject comprises one or more of the SNPs Rs6152, Rs 2223841, Rs2497938, Rs1160312, 6047844, Rs2180439, Rs2073963, Rs12565727, Rs9287638, Rs6945541, Rs12373124, Rs10502861, Rs187238, and/or Rs1946518.

47. The method of claim 44, wherein the cancer comprises pancreatic cancer or breast cancer.

48. The method of claim 47, wherein the disease is breast cancer and the SNP associated with the breast cancer in the subject comprises the SNP Rs2298075.

49. The method of claim 47, wherein the disease is pancreatic cancer and the SNP associated with the pancreatic cancer in the subject comprises the SNP Rs10237038.

50. The method of claim 44, wherein the disease or disorder comprises autism spectrum disorder and the SNP associated with the autism spectrum disorder comprise one or more of the SNPs Rs237887, Rs 2268491, Rs2254298, and/or Rs 7632287.

51. The method of claim 44, wherein the disease or disorder comprises osteoporosis and the SNP associated with the osteoporosis comprises one or more of the SNPs Rs28360447, Rs28360457, Rs1718119, Rs2230911, and/or Rs1653624.

52. The method of claim 44, wherein the disease or disorder comprises Rheumatoid Arthritis and the SNP associated with the Rheumatoid Arthritis comprises the SNP Rs2269706.

53. The method of claim 44, wherein the disease or disorder comprises Multiple Sclerosis and the SNP associated with the Multiple Sclerosis comprises one or more of the SNPs Rs17525809 and/or Rs28360447.

54. The method of claim 44, wherein the disease or disorder comprises chronic pain and the SNP associated with the chronic pain comprises one or more of the SNPs Rs28360447 and/or Rs7958311.

55. The method of claim 44, wherein the disease or disorder comprises atopic dermatitis and the SNP associated with the atopic dermatitis comprises the SNP Rs6010620.

56. The method of claim 44, wherein the disease or disorder comprises hypertension and the SNP associated with the hypertension comprises the SNP Rs11669309.

57. The method of claim 44, wherein the disease or disorder comprises coronary heart disease and the SNP associated with the heart disease comprises the SNP Rs6982502.

58. The method of claim 44, wherein the disease or disorder comprises type II diabetes and the SNP associated with the type II diabetes comprises the SNP Rs12683158.

59. The method of claim 44, wherein the disease or disorder comprises celiac disease and the SNP associated with the celiac disease comprises the SNP Rs15428265.

60. The method of claim 44, wherein the disease or disorder anxiety and the SNP associated with the anxiety comprises the SNP Rs1718119.

61. The method of claim 44, wherein the disease or disorder comprises bipolar disorder and the SNP associated with the bipolar disorder comprises the SNP Rs2230912.

62. The method of claim 44, wherein the disease or disorder comprises depression and the SNP associated with the depression comprises the SNP Rs8028149.

63. The method of claim 44, wherein the disease or disorder comprises the one or more of the SNPs Rs16868911, Rs11543230, Rs9309325, and/or Rs12951337.
